(19) United States
(12) Patent Application Publication
(10) Pub. No.: US 2012/0158143 A1
(43) Pub. Date: Jun. 21, 2012

(54) ALIF Spinal Cage

(76) Inventor: Gary S. Shapiro, Glenview, IL (US)

(21) Appl. No.: 13/330,898

(22) Filed: Dec. 20, 2011

Related U.S. Application Data
(60) Provisional application No. 61/424,863, filed on Dec. 20, 2010.

(51) Int. Cl. A61F 2/44

(52) U.S. Cl. 623/17.16

(57) ABSTRACT

A fusion cage having a substantially continuous sidewall made from any of various materials including titanium, PEEK, a carbon graphite fiber, or even a combination of these materials. A hollow core defined by the sidewall is typically filled with bone graft in order to form a solid fusion with vertebrae. The sidewall of the cage has upper and lower surfaces, an anterior section, at least two lateral sections each connected to the anterior section, and a posterior section connected to the lateral sections. The sidewall is preferably tapered at one of either a four or eight degree incline from the posterior to the anterior section so as to maintain backbone curvature. A plurality of distinct tool ports provide for anterior, lateral and/or anterolateral insertion of the device. Each tool port is provided with screw insertion orifices.

### RELATED APPLICATION

The present utility application claims the earlier filing priority of U.S. Provisional Application No. 61/424,863, titled “ALIF SPINAL CAGE” and filed on Dec. 20, 2010 (the '863 application). The entirety of the '863 application is hereby incorporated by reference.

### TECHNICAL FIELD OF THE INVENTION

The present device relates to a non-threaded spinal cage for use in maintaining an adequate disc height after Anterior Lumbar Interbody Fusion (ALIF) surgery. Particularly, the present device relates to an ALIF cage having multiple insertion capabilities as well as multiple screw fixations capabilities. Further, the device relates to an ALIF surgical system which employs an ALIF cage. The surgical system includes the disclosed ALIF cage as well as suitable surgical tools, such as an inserter/distractor, and is useful in either an open surgical procedure or a minimally invasive procedure.

### BACKGROUND OF THE INVENTION

The Anterior Lumbar Interbody Fusion (ALIF) surgical procedure, using devices such as cages and bone dowels, has been in use for over ten years. The intervertebral fusion cage is another tool for the spine surgeon to use in helping treat various low back problems. The fusion cage is a device which is inserted by the surgeon between two vertebra of a patient to relieve stresses and other causes of back pain and discomfort. Typically, a strong polymer material such as polyether-ether-ketone (PEEK), is common for such cages. The benefits of this material are well-known by those in the medical field.

For patients that require fusion surgery to treat such ailments as degenerative disc disease, deformity and instability, the ALIF cage has been shown to be effective for several reasons, including:
wide surface area enabling high fusion rates;
successful correction of deformity;
restoration of disc height; and
avoidance of neurological elements.

The ALIF cage device may act as a stand-alone device to promote fusion and maintain disc height without the need for posterior surgery and instrumentation of the spine, but it may also work in conjunction with surgery and instrumentation in some cases.

Prior art cages can usually be implanted from either the front or the back of the spine. Insertion from the front requires entry through the patient's abdomen. Surgery from the back of the spine may be used when, for example, the removal of bone spurs or a herniated disc is needed. In such instances, the cages can be implanted from the patient's back, without having to make an additional incision in the patient's abdomen.

The surgical process of inserting a cage through the front of the spine is called anterior insertion. Working from the front can be done with an open procedure, where an incision is made through the patient's abdomen. This procedure usually requires preparation of the patient by a general surgeon before cage insertion by a spine surgeon. Alternative insertion techniques include minimally invasive lateral insertion—requiring an incision in the patient's side—and anterior-lateral insertion—requiring an incision through a point lateral to the anterior insertion point and anterior to the lateral insertion point. The spine surgeon can determine which of the insertion techniques is best suited for a particular patient.

Despite the insertion options, many cages are only capable of a single insertion technique, most particularly anterior insertion. The drawbacks of single-technique devices may be significant. For example, a surgeon may have to commit to an insertion technique based on the cage device at hand. Also, a particular cage may not have a suitable insertion/distraction tool on the market. This is problematic in that insertion of the cage can require a fair amount of force and the cage or vertebral bone can become damaged in the process. Without the use of an inserter/distractor, insertion and removal of a damaged cage can be difficult and time consuming. Even further, some of the cages on the market are available only in PEEK and carbon fiber, making them more prone to damage when impacted.

No device or surgical method in use at the present time overcomes the various problems associated with prior art ALIF cages. It would be desirable if the benefits of a single, ALIF surgery could be obtained with a strong, sturdy cage capable of use in any of the preferred insertion techniques, the ability to use an inserter/distractor, as well as being capable of the three corresponding screw fixations to prevent cage migration. The present invention addresses these and other problems associated with prior art devices.

### BRIEF DESCRIPTION OF THE DRAWINGS

For the purpose of facilitating an understanding of the subject matter sought to be protected, there are illustrated in the accompanying drawings embodiments thereof, from an inspection of which, when considered in connection with the following description, the subject matter sought to be protected, its construction and operation, and many of its advantages should be readily understood and appreciated.

FIG. 1 is a perspective view from one side of an embodiment of the present device;

FIG. 2 is a perspective view from another side of the embodiment shown in FIG. 1;

FIG. 3 is a bottom view of the embodiment shown in FIG. 1;

FIG. 4 is a front view of the embodiment shown in FIG. 1;

FIG. 5 is a side view of the embodiment shown in FIG. 1;

FIG. 6 is another side view of the embodiment shown in FIG. 1;

FIG. 7 is a rear view of the embodiment shown in FIG. 1;

FIG. 8 is a perspective view of an embodiment of the present device illustrating placement of three different fixation screws sets;

FIG. 9 is a perspective view of another embodiment (Titanium body) of the present device;

FIG. 10 is a perspective view similar to that of FIGS. 1 and 2, except only two tool ports are provided;

FIG. 11 is a perspective view similar to that of FIGS. 1 and 2, except all three tool ports are provided contiguously;

FIG. 12 is a perspective view similar to that of FIGS. 1 and 2, except only two contiguous ports are provided;

FIG. 13 is a side view of a tool useful in placement of a cage; and

FIG. 14 is a perspective view of a tool useful in placement of a cage, the end of the tool being magnified to show detail and indicate attachment of a cage.

### DETAILED DESCRIPTION OF PREFERRED EMBODIMENTS

While this invention is susceptible of embodiments in many different forms, there is shown in the drawings and will herein be described in detail a preferred embodiment of the invention with the understanding that the present disclosure is to be considered as an exemplification of the principles of the invention and is not intended to limit the broad aspect of the invention to embodiments illustrated.

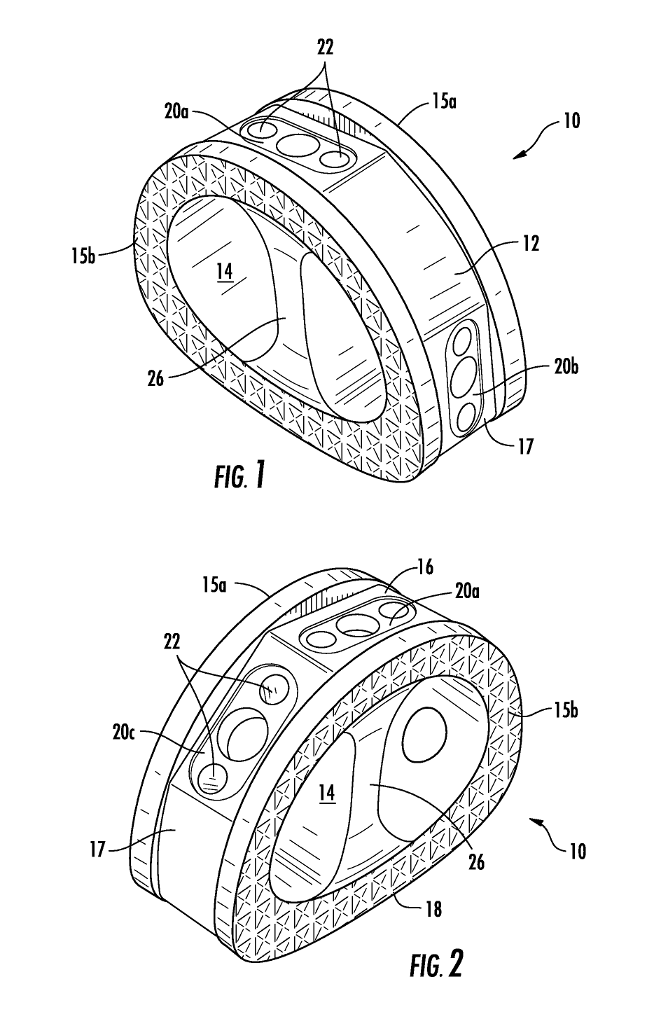

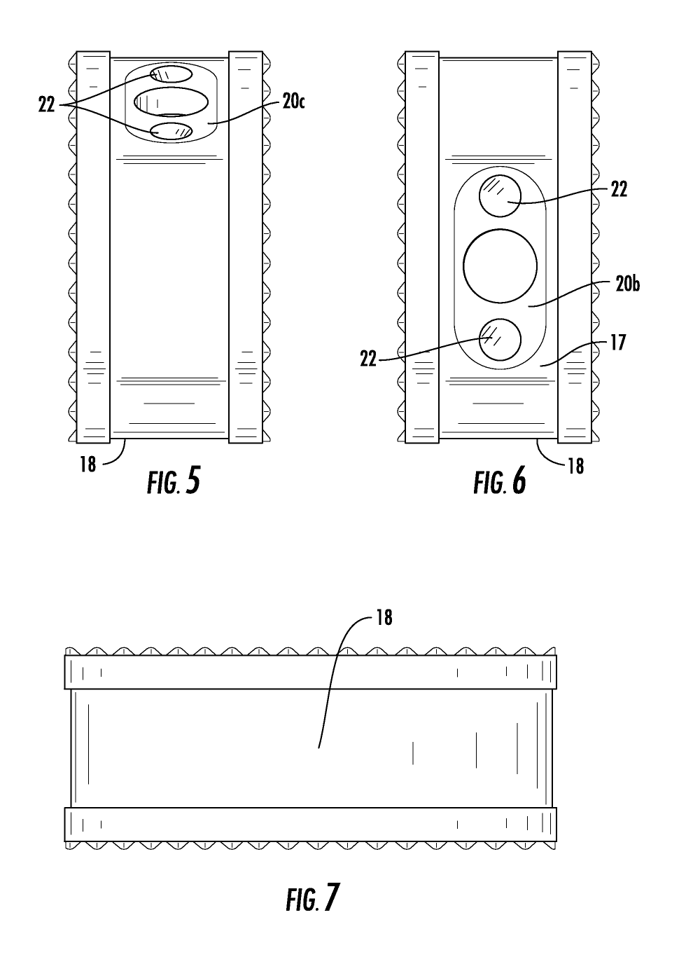

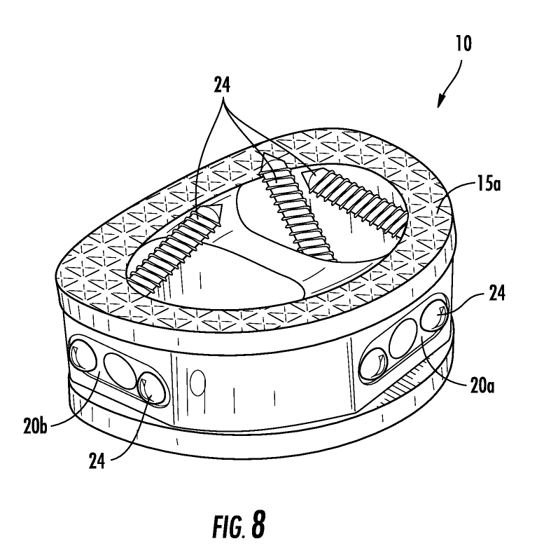

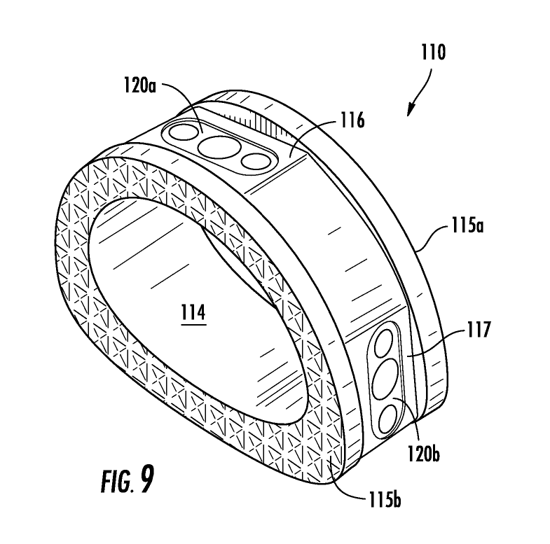

Referring to FIGS. 1-14, there are illustrated embodiments of a fusion cage, generally designated by the numeral 10. The cage 10 includes a substantially continuous sidewall 12 made from any of various materials including titanium, PEEK, carbon graphite fiber, carbon fiber reinforced polymer (CFRP) or even a combination of these materials. A hollow core 14 is defined by the sidewall 12 and is typically used for filling with bone graft (not shown) by the surgeon before insertion. The open core 14 keeps the graft material in contact with the bony surface of the vertebrae for increased fusion success. This configuration ensures that the bone graft unites with the vertebrae, forming a solid fusion. The use of bone graft is well-known and understood by those skilled in the art.

The sidewall 12 of the cage 10 has an upper surface 15a and a lower surface 15b, an anterior section 16, at least two lateral sections 17 each connected to the anterior section 16, and a posterior section 18 connected to each of the at least two lateral sections 17. The sidewall 12 is preferably tapered at one of either a four degree (4°) or eight degree (8°) incline from the posterior section 18 toward the anterior section 16 so as to maintain a curvature of the backbone (lordosis). The upper and lower surfaces 15a,b are preferably textured to promote grafting and retention of the device 10 within a created space. The texturing may include cross-hatching (see FIG. 3), serrations, or any suitable regular or random texturing pattern suitable for the purposes described herein. A coating of porous material may also be applied to each of the upper and lower surfaces 15a,b of the continuous sidewall 12 to further promote adhesion.

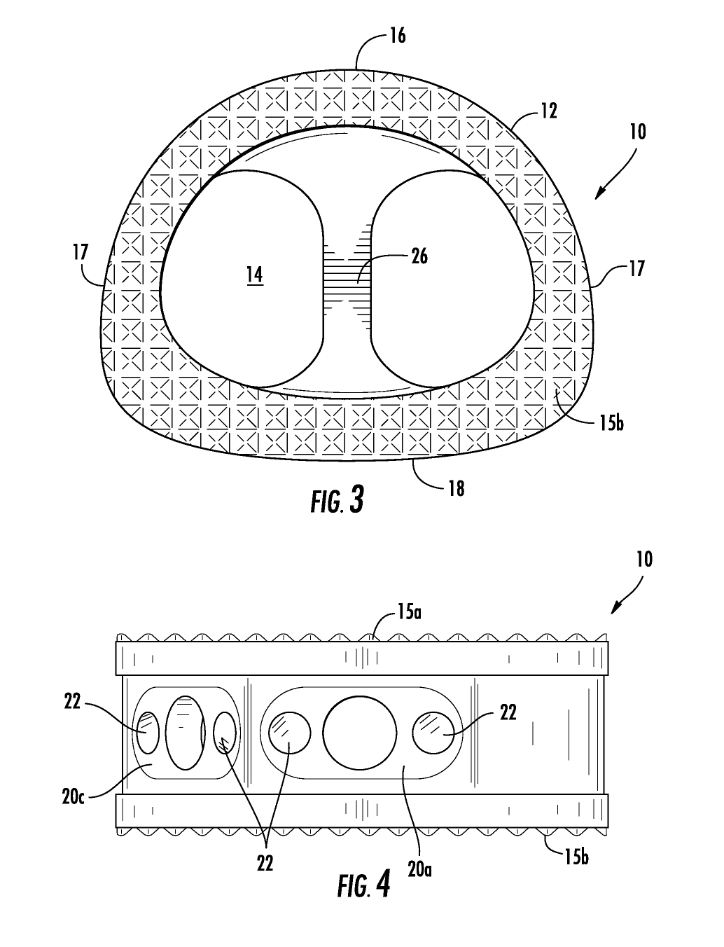

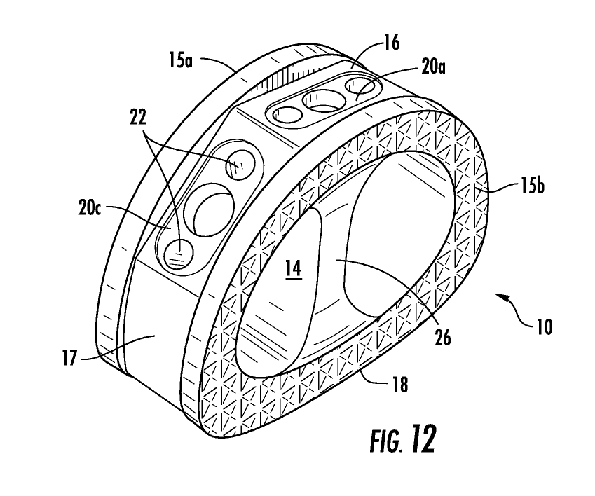

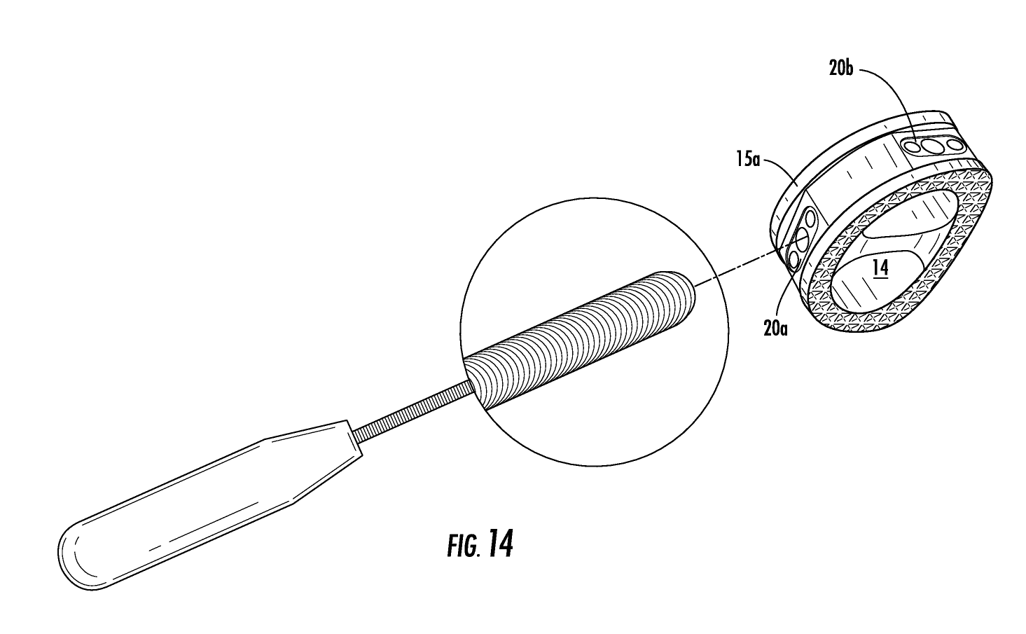

The disclosed cage 10 is comprised of a plurality of tool ports for varied insertion procedures. As shown in FIGS. 1 and 2, three distinct tool ports 20a-c provide the opportunity for anterior, lateral and anteriolateral insertion of the cage 10 into the patient. The surgery for insertion of the cage 10 may be done as an open surgical procedure or as a minimally invasive surgical procedure. FIGS. 10 and 12 illustrate embodiments where two distinct tool ports—20a and 20b in FIGS. 10 and 20a and 20c in FIG. 12—are provided. The tool port 20 is comprised of an opening 23 which allows attachment of the cage 10 to an insertion tool end (see FIG. 14). The opening 23 is preferably threaded to allow the cage to be screwed onto the tool end, but the connection may be constructed in any manner which allows secure attachment of the cage 10 to the insertion tool (e.g., friction fit). Such alternate attachment means are currently in use with prior art cages.

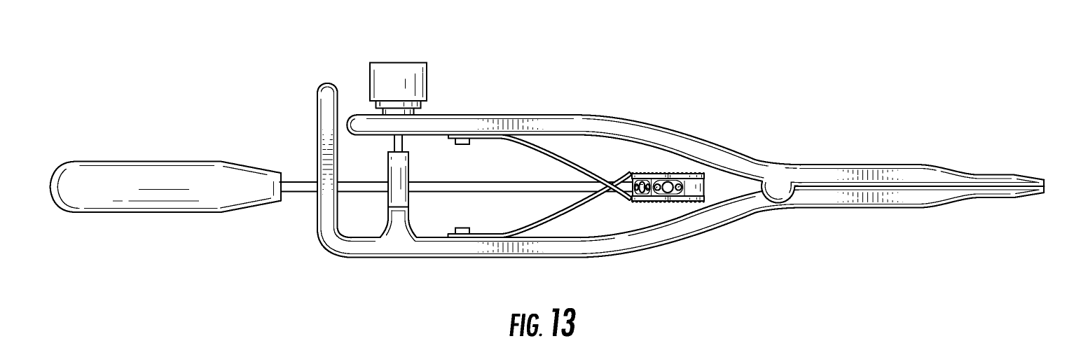

In the disclosed embodiments, a first insertion tool port 20a is positioned within the anterior section 16 of the sidewall 12, a second insertion tool port 20b is positioned within one of either lateral sections 17 of the sidewall 12, and a third insertion tool port 20c is positioned in the sidewall 12 between the first insertion tool port 20a and a lateral section 17. As shown in FIG. 11, the tool ports 20a-c may be contiguous, though this embodiment may not be preferred from a structural integrity standpoint. Also, where only two tool ports are provided, three alternate arrangements of the ports are possible (i.e., first and second, first and third, and second and third).

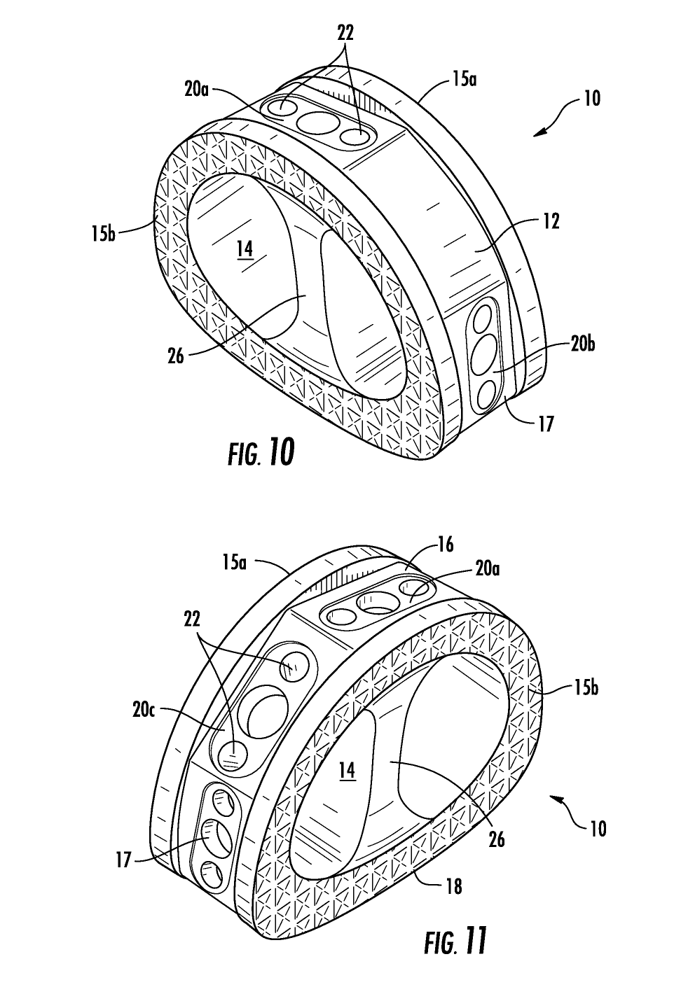

Each of the insertion tool ports 20a-c is provided with adjacent screw insertion orifices 22. As shown in FIG. 8, the orifices 22 are provided at angles to allow one screw 24 to angle upward and another screw 24 to angle downward. This is an accepted attachment method used by those skilled in the art. However, alternate screw configurations are possible, provided the orifices 22 do not interfere with the tool ports 20.

The screw orifices 22 are preferably provided with a feature to prevent screw 24 from backing out over time. The myriad of alternatives for providing this anti-loosening feature (not shown) include a pivoting or sliding interference cam, locking rings, and any device which inhibits the screw from exiting the cage orifice 22. Of course, the interference must be capable of being removed by a surgeon should, for example, the cage need to be removed for any reason.

The vertebral body fusion cage 10 is preferably comprised of one of either titanium, polyether-ether-ketone (PEEK), a composite, or any combination of the same. Titanium offers the greatest strength, while the PEEK is a more cost-effective option. By combining the materials, a hybrid cage having high structural integrity as well as being cost-effective, can be produced. For example, manufacturing the cage predominately of PEEK material while adding titanium formed tool ports and screw orifices provides a much stronger anchoring material.

Additionally, to improve the structural integrity of the PEEK device, a third lateral section 26 connected to each of the anterior section 16 and the posterior section 18 of the sidewall 12, passing through the hollow core 14 proximate a midline may be provided on embodiments of the device 10. The titanium embodiment is illustrated best in the cage 110 of FIG. 9. The stronger titanium version would be suitable without the reinforcing lateral section 26 in core 114, but has all the remaining elements (e.g., ports 120a-c, anterior section 116, lateral sections 117, posterior section 118, etc.).

In an Anterior Lumbar Interbody Fusion (ALIF) surgical assembly, each of the individually above-described embodiments of the cage 10 may be provided with an inserter/distractor 30 which detachably couples to the insertable cage 10, as shown in FIGS. 13 and 14. The inserter/distractor 30 is used to space adjacent vertebrae at the insertion site and deliver the cage 10 to the insertion site. This is well-understood by those skilled in the art. The inserter/distractor 30 may be comprised of two separate tools, as shown, or a single tool which provides both features. Other embodiments of the assembly might include screws 24 for attaching the cage 10 to a vertebral body. Additional optional instruments for embodiments of the disclosed assembly might include at least one instrument selected from osteotomers, curettes, Rongeurs, trials, and paddle distractors. These instruments are well-known by those skilled in the art.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. A vertebral body fusion cage comprising:
a sidewall having upper and lower surfaces, an anterior section, at least two lateral sections each adjacent the anterior section, and a posterior section adjacent each of the at least two lateral sections; and
a plurality of distinct insertion tool ports.

2. The vertebral body fusion cage of claim 1, wherein the sidewall tapers at an angle from the anterior section to the posterior section.

3. The vertebral body fusion cage of claim 1, wherein the sidewall is comprised of titanium metal.

4. The vertebral body fusion cage of claim 3, further comprising a porous coating applied to each of the upper and lower surfaces of the sidewall.

5. The vertebral body fusion cage of claim 1, wherein the number of distinct insertion tool ports is either two or three.

6. The vertebral body fusion cage of claim 5, wherein the number of distinct insertion tool ports is three and the tool ports comprise:
a first insertion tool port positioned on the anterior section of the sidewall;
a second insertion tool port positioned on one of either lateral sections of the sidewall; and
a third insertion tool port positioned on the sidewall between the first insertion tool port and a lateral section of the sidewall.

7. The vertebral body fusion cage of claim 5, wherein the number of distinct insertion tool ports is two and the tool ports comprise:
a first insertion tool port positioned on the anterior section of the sidewall; and
an additional tool port positioned at one of either a lateral sections of the sidewall or between the first insertion tool port and a lateral section of the sidewall.

8. The vertebral body fusion cage of claim 1, wherein the sidewall is comprised of a material selected from one of either polyether-ether-ketone (PEEK), titanium, a composite, and any combination of the same.

9. The vertebral body fusion cage of claim 1, further comprising orifices in the sidewall for the insertion of screws.

10. The vertebral body fusion cage of claim 1, wherein the cage is non-threaded.

11. A vertebral body fusion cage comprising:
a continuous sidewall having upper and lower surfaces, an anterior section, at least two lateral sections each connected to the anterior section, and a posterior section connected to each of the at least two lateral sections;
a first insertion tool port positioned within the anterior section of the sidewall;
at least one additional insertion tool port positioned within one of either a lateral section of the sidewall or between the first insertion tool port and either lateral section of the sidewall; and
a porous coating applied to each of the upper and lower surfaces of the continuous sidewall to promote adhesion; wherein the sidewall tapers at an angle from the anterior section to the posterior section.

12. The vertebral body fusion cage of claim 11, wherein the sidewall is comprised of a material selected from one of either polyether-ether-ketone (PEEK), titanium, a composite, and any combination of the same.

13. The vertebral body fusion cage of claim 11, wherein the sidewall is comprised of titanium.

14. The vertebral body fusion cage of claim 11, further comprising orifices in the sidewall for the insertion of screws.

15. The vertebral body fusion cage of claim 11, further comprising a third lateral section connected to each of the anterior section and the posterior section of the sidewall proximate a midline.

16. The vertebral body fusion cage of claim 11, wherein the cage is non-threaded.

17. The vertebral body fusion cage of claim 11, wherein the number of insertion tool ports is three.

18. An Anterior Lumbar Interbody Fusion (ALIF) surgical assembly comprising:
an insertable cage comprising a continuous sidewall having upper and lower surfaces, an anterior section, at least two lateral sections each connected to the anterior section, and a posterior section connected to each of the at least two lateral sections, wherein the sidewall tapers at an angle from the anterior section to the posterior section;
a first insertion tool port positioned within the anterior section of the sidewall;
at least one additional insertion tool port positioned within one of either a lateral section of the sidewall or between the first insertion tool port and either of the lateral sections of the sidewall;
a porous coating applied to each of the upper and lower surfaces of the continuous sidewall to promote adhesion; and
an inserter/distractor which detachably couples to the insertable cage.

19. The Anterior Lumbar Interbody Fusion assembly of claim 18, further comprising screws for attaching the cage to a vertebral body.

20. The Anterior Lumbar Interbody Fusion assembly of claim 18, further comprising at least one instrument selected from osteotomers, curettes, Rongeurs, trials, and paddle distractors.
